(19) United States
(12) Patent Application Publication
(10) Pub. No.: US 2005/0210562 A1
(43) Pub. Date: Sep. 29, 2005

(54) Game call retention apparatus

(76) Inventors: Steve M. Johnson, Church Hill, TN (US); Darrell R. Mullins, Bluff City, TN (US)

Correspondence Address: S. Michael Bender, P.O. Box 530399, St. Petersburg, FL, 33747, US

(21) Appl. No.: 10/808,976

(22) Filed: Mar. 25, 2004

(51) Int. Cl. A61F 9/00; A41D 19/00

(52) U.S. Cl. 2/159

(57) ABSTRACT

An article retention apparatus is provided which includes a glove portion, first article retention means which include first retainer closure means, and glove-to-retainer connection means for connecting the glove portion to the first article retention means. The glove-to-retainer connection means are located so as to enable positioning of the first article retention means on a back side of the glove portion. With one embodiment, the glove-to-retainer connection means include a pouch-mounted hook-or-loop connector attached to a back side of the first article retention pouch and include a complementary glove-mounted loop-or-hook connector attached to the glove back side. With another embodiment, the glove-to-retainer connection means include flexible hinge members connected between the glove portion and a combination of pouches, enabling the combination of pouches to be readily flipped from the glove back side to the glove palm side and vice versa.

### BACKGROUND OF THE INVENTION

1. Field of the Invention

The present invention relates generally to article holders, and, more particularly, to an article holder especially adapted for retaining game call apparatus.

2. Description of the Prior Art

Game calls are often used by hunters to attract game. For example, a turkey call can consist of two parts, a call member and a friction peg. When out in the field, the hunter usually carries the call member and the friction peg in a shirt, coat, or pants pocket. To use the call apparatus, the hunter often has to remove one's gloves and dig into the respective pockets to bring out the game call apparatus. In cold weather, not only is it inconvenient for the hunter to have to remove one's gloves to access the game call apparatus, but the weather may be cold, and the hunter's hands may become cold or freeze when the gloves are removed. In this respect, it would be desirable if a game call retention apparatus were provided than enabled a hunter to access a game call apparatus without removing one's gloves.

There are a number of types of game call apparatus available to the hunter. Aside from the type of apparatus mentioned above, there are also mouth calls. To provide a hunter with easy choice among game call apparatuses, it would be desirable if a game call retention apparatus were provided which can retain a plurality of different types of game call apparatuses.

There are times when a hunter has no interest in employing a game call apparatus. During such times, it would be desirable if a game call retention apparatus were provided that can be readily separated from the hunter.

As mentioned above, it is common for a hunter to carry a game call apparatus in a pocket in one's shirt, coat, or pants. Then, to use the game call apparatus, the hunter must remove the game call apparatus from the respective pocket. Digging a game call apparatus out from one's pocket can be very inconvenient. For example, if the game call apparatus is carried in a shirt pocket, then the hunter may have to unzip one's coat in order to access one's shirt pocket. To avoid such inconveniences, it would be desirable if a game call retention apparatus were provided which precludes the need for a hunter to store a game call apparatus in one's pocket.

Since some game call apparatus are used in a hunter's hands, for convenience to the hunter, it would be desirable if a game call retention apparatus were provided that is located in the close vicinity of the hunter's hands.

As mentioned above, a hunter must often dig a game call apparatus out from one's pocket in order to use the apparatus. More generally, a hunter would have to dig a game call apparatus out from a game call retention apparatus. To avoid the need for a hunter to have to dig a game call apparatus out from a game call retention apparatus, it would be desirable if a game call retention apparatus were provided which enabled a hunter to use the game call retention apparatus without removing the game call apparatus from a retention apparatus.

Thus, while the foregoing indicates it to be well known to use game call apparatuses, the foregoing does not teach or suggest a game call retention apparatus which has the following combination of desirable features: (1) enables a hunter to access a game call apparatus without removing one's gloves; (2) can retain a plurality of different types of game call apparatuses; (3) can be readily separated from the hunter; (4) precludes the need for a hunter to store a game call apparatus in a conventional garment pocket; (5) is located in the close vicinity of the hunter's hands; and (6) enables a hunter to use the game call retention apparatus without removing the game call apparatus from the retention apparatus. The foregoing desired characteristics are provided by the unique game call retention apparatus of the present invention as will be made apparent from the following description thereof. Other advantages of the present invention over the prior art also will be rendered evident.

### SUMMARY OF THE INVENTION

To achieve the foregoing and other advantages, the present invention, briefly described, provides an article retention apparatus is provided which includes a glove portion, first article retention means which include first retainer closure means, and glove-to-retainer connection means for connecting the glove portion to the first article retention means. The glove-to-retainer connection means are located so as to enable positioning of the first article retention means on a back side of the glove portion.

With one embodiment of the invention, the glove-to-retainer connection means include stitches between the first article retention means and the back side of the glove portion. The first article retention means include a first article retention pouch, and the first retainer closure means include a first retainer closure elastic band located at an opening of the first article retention pouch.

With another embodiment of the invention, the glove-to-retainer connection means include a pouch-mounted hook-or-loop connector attached to a back side of the first article retention pouch and include a complementary glove-mounted loop-or-hook connector attached to the glove back side. In addition, the first retainer closure means is made from closure hook-or-loop material.

Second article retention means are connected to the first article retention means. The second article retention means include a second article retention pouch. The second article retention pouch includes a plurality of pouch compartments. Each of the pouch compartments includes an open top end and an open bottom end. The pouch compartments are used for receiving friction pegs.

With another embodiment of the invention, a third article retention pouch is connected between the first article retention pouch and the second article retention pouch.

The glove-to-retainer connection means include flexible hinge members which are connected between the glove portion and the first article retention pouch. The flexible hinge members are connected to the glove portion at hinge rotation regions. The flexible hinge members are connected to a side edge of the glove portion. By using the flexible hinge members, a combination of the first, second, and third pouches can be readily flipped from the glove back side to the glove palm side and vice versa.

In addition, lock means are provided for locking the first article retention pouch to a back side of the glove portion. The lock means include a lock tab. The lock tab includes a quantity of a hook-or-loop connector which engages a quantity of complementary loop-or-hook connector on the glove portion for locking the lock tab to the glove portion.

The first article retention pouch includes an elastic frame for retaining an exposed call member which can be struck or scratched without removing the call member from the first article retention pouch.

The above brief description sets forth rather broadly the more important features of the present invention in order that the detailed description thereof that follows may be better understood, and in order that the present contributions to the art may be better appreciated. There are, of course, additional features of the invention that will be described hereinafter and which will be for the subject matter of the claims appended hereto.

In this respect, before explaining at least three preferred embodiments of the invention in detail, it is understood that the invention is not limited in its application to the details of the construction and to the arrangements of the components set forth in the following description or illustrated in the drawings. The invention is capable of other embodiments and of being practiced and carried out in various ways. Also, it is to be understood, that the phraseology and terminology employed herein are for the purpose of description and should not be regarded as limiting.

As such, those skilled in the art will appreciate that the conception, upon which disclosure is based, may readily be utilized as a basis for designing other structures, methods, and systems for carrying out the several purposes of the present invention. It is important, therefore, that the claims be regarded as including such equivalent constructions insofar as they do not depart from the spirit and scope of the present invention.

It is therefore an object of the present invention to provide a new and improved game call retention apparatus which has all of the advantages of the prior art and none of the disadvantages.

It is another object of the present invention to provide a new and improved game call retention apparatus which may be easily and efficiently manufactured and marketed.

It is a further object of the present invention to provide a new and improved game call retention apparatus which is of durable and reliable construction.

An even further object of the present invention is to provide a new and improved game call retention apparatus which is susceptible of a low cost of manufacture with regard to both materials and labor, and which accordingly is then susceptible of low prices of sale to the consuming public, thereby making such game call retention apparatus available to the buying public.

Still yet a further object of the present invention is to provide a new and improved game call retention apparatus which enables a hunter to access a game call apparatus without removing one's gloves.

Still another object of the present invention is to provide a new and improved game call retention apparatus that can retain a plurality of different types of game call apparatuses.

Yet another object of the present invention is to provide a new and improved game call retention apparatus which can be readily separated from the hunter.

Even another object of the present invention is to provide a new and improved game call retention apparatus that precludes the need for a hunter to store a game call apparatus in a conventional garment pocket.

Still a further object of the present invention is to provide a new and improved game call retention apparatus which is located in the close vicinity of the hunter's hands.

Yet another object of the present invention is to provide a new and improved game call retention apparatus that enables a hunter to use the game call retention apparatus without removing the game call apparatus from the retention apparatus.

These together with still other objects of the invention, along with the various features of novelty which characterize the invention, are pointed out with particularity in the claims annexed to and forming a part of this disclosure. For a better understanding of the invention, its operating advantages and the specific objects attained by its uses, reference should be had to the accompanying drawings and descriptive matter in which there are illustrated preferred embodiments of the invention.

### BRIEF DESCRIPTION OF THE DRAWINGS

The invention will be better understood and the above objects as well as objects other than those set forth above will become more apparent after a study of the following detailed description thereof. Such description makes reference to the annexed drawing wherein:

FIG. 1 is a top view showing a first embodiment of the game call retention apparatus of the invention which includes a first article retention pouch which includes a first retainer closure elastic band and which includes stitches for connecting the first article retention pouch to the glove portion.

FIG. 2 is a cross-sectional view of the embodiment of the game call retention apparatus shown in FIG. 1 taken along line 2-2 of FIG. 1.

FIG. 3 is a top view of a second embodiment of the game call retention apparatus of the invention which includes a first article retention pouch, a second article retention pouch which includes a plurality of pouch compartments, and complementary hook-and-loop connectors for connecting the first article retention pouch to the glove portion.

FIG. 4 is a cross-sectional view of the embodiment of the game call retention apparatus shown in FIG. 3 taken along line 4-4 of FIG. 3.

FIG. 5 is an exploded perspective view of a second embodiment of the invention shown in FIGS. 3 and 4.

FIG. 6 is a top view of a third embodiment of the game call retention apparatus of the invention which includes a first article retention pouch, a second article retention pouch which includes a plurality of pouch compartments, a third article retention pouch between the first article retention pouch and the second article retention pouch, and flexible hinge members which connect the first article retention pouch to the glove portion, wherein the first article retention pouch is in contact with the glove back side.

FIG. 7 is a side view of the embodiment of the invention shown in FIG. 6, taken along line 7-7 thereof, wherein the first article retention pouch and the second article retention pouch have been rotated around the flexible hinge members so that the second article retention pouch rests against the glove palm side.

FIG. 8 a top view of the embodiment of the invention shown in FIG. 6, wherein the first, second, and third article retention pouches are shown in broken lines on the glove back side, and wherein the first, second, and third article retention pouches are shown in solid lines on the glove palm side.

FIG. 9 is a view of the third embodiment of the invention from the glove palm side showing that the first article retention pouch includes an elastic frame for retaining an exposed call member.

### DESCRIPTION OF THE PREFERRED EMBODIMENT

With reference to the drawings, a new and improved game call retention apparatus embodying the principles and concepts of the present invention will be described.

An article retention apparatus 10 is provided which includes a glove portion 12, first article retention means which include first retainer closure means, and glove-to-retainer connection means for connecting the glove portion 12 to the first article retention means. The glove-to-retainer connection means are located so as to enable positioning of the first article retention means on a back side of the glove portion 12.

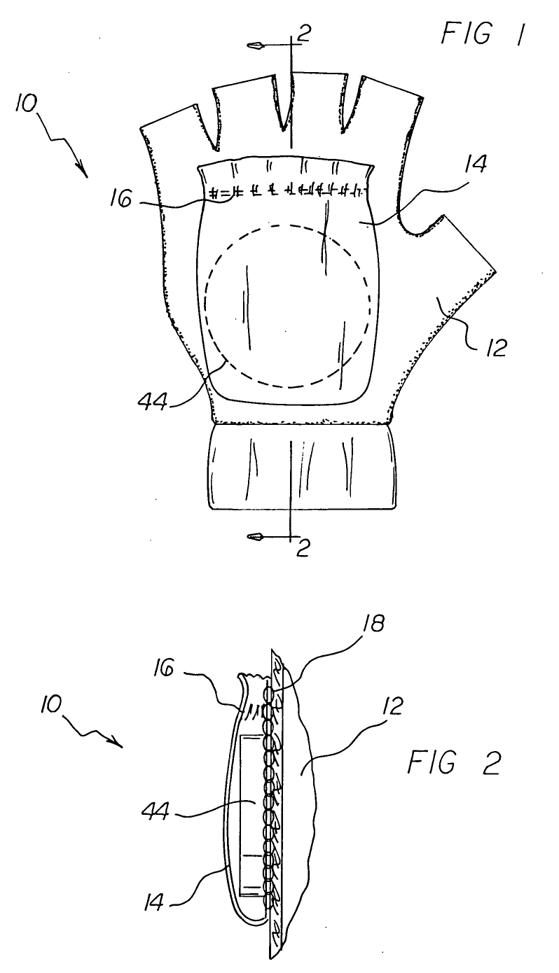

As shown with the first embodiment 10 of the invention shown in FIGS. 1 and 2, the glove-to-retainer connection means include stitches 18 between the first article retention means and the back side of the glove portion 12. The first article retention means include a first article retention pouch 14, and the first retainer closure means include a first retainer closure elastic band 16 located at an opening of the first article retention pouch 14.

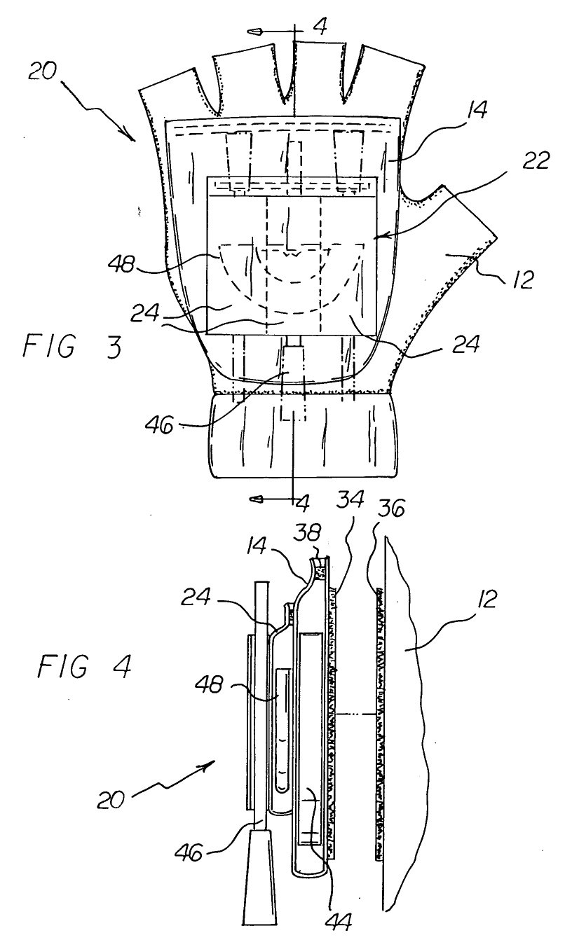

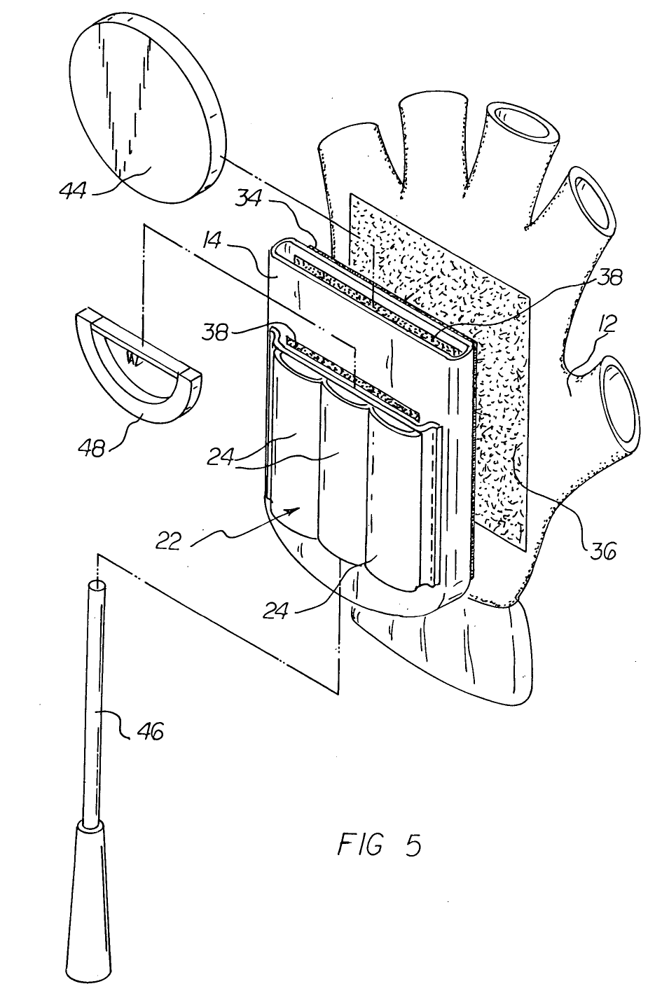

As shown in FIGS. 1 and 2, the first article retention pouch 14 can be used for storing a call member 44 that is to be sounded by hitting or scratching with a friction peg 46 (shown in FIGS. 3-5).

To use the first embodiment of the invention, the first retainer closure elastic band 16 is stretched open, and the call member 44 is removed from the first article retention pouch 14. Then, a friction peg 46 can be used to strike or scratch the call member 44. When the call member 44 is no longer needed, the call member 44 is returned to the first article retention pouch 14.

Turning to FIGS. 3-5, a second embodiment 20 of the invention is shown. Reference numerals are shown that correspond to like reference numerals that designate like elements shown in the other figures. In addition, The glove-to-retainer connection means include a pouch-mounted hook-or-loop connector 34 attached to a back side of the first article retention pouch 14 and include a complementary glove-mounted loop-or-hook connector 36 attached to the glove back side. In addition, the first retainer closure means is made from closure hook-or-loop material 38.

Second article retention means are connected to the first article retention means. The second article retention means include a second article retention pouch 22. The second article retention pouch 22 includes a plurality of pouch compartments 24. Each of the pouch compartments 24 includes an open top end and an open bottom end. The pouch compartments 24 are used for receiving friction pegs 46.

A third article retention pouch 26 is connected between the first article retention pouch 14 and the second article retention pouch 22. As shown in FIGS. 3-5, the third article retention pouch 26 can receive a mouth call 48.

As shown in FIGS. 3-5, the first article retention pouch 14, the second article retention pouch 22, and the third article retention pouch 26 can be formed as a combination of the first, second, and third pouches. When the combination of the first, second, and third pouches is to be used for retaining various articles, the pouch-mounted hook-or-loop connector 34 is connected to the glove-mounted loop-or-hook connector 36 to attach the combination of the first, second, and third pouches to the glove portion 12. The pouch-mounted hook-or-loop connector 34 and the glove-mounted loop-or-hook connector 36 can be made from well-known VELCRO™ material.

On the other hand, if the combination of the first, second, and third pouches and articles retained therein are not to be carried by the glove portion 12, then the pouch-mounted hook-or-loop connector 34 is separated from the glove-mounted loop-or-hook connector 36, and the glove portion 12 is free of the combination of the first, second, and third pouches and articles therein.

As shown in FIGS. 3-5, the first article retention pouch 14 can contain a call member 44. The second article retention pouch 22 can contain a plurality of friction pegs 46 in the respective pouch compartments 24. The third article retention pouch 26 can contain a mouth call 48.

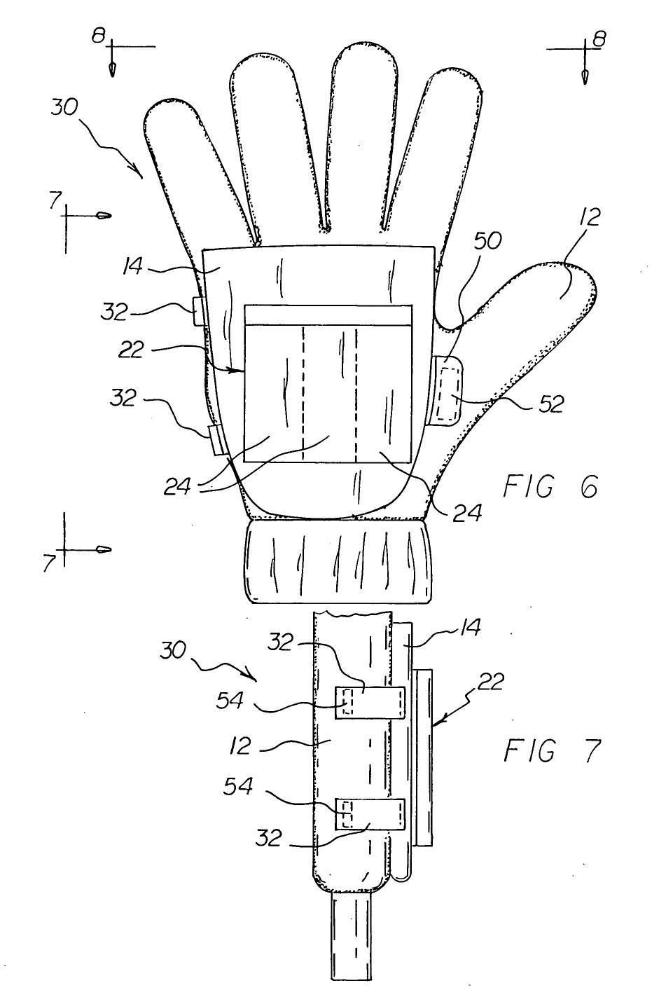

As shown in the third embodiment 30 in FIGS. 6-9, the glove-to-retainer connection means include flexible hinge members 32 which are connected between the glove portion 12 and the first article retention pouch 14. The flexible hinge members 32 are connected to the glove portion 12 at hinge rotation regions 54. The flexible hinge members 32 are connected to a side edge of the glove portion 12.

In addition, lock means are provided for locking the first article retention pouch 14 to a back side of the glove portion 12. The lock means include a lock tab 50. The lock tab 50 includes a quantity of a hook-or-loop connector 52 which engages a quantity of complementary loop-or-hook connector on the glove portion 12 for locking the lock tab 50 to the glove portion 12.

The first article retention pouch 14 includes an elastic frame 42 for retaining an exposed call member 44.

To use the third embodiment 30 of the invention shown in FIGS. 6-9, the pouch compartments 24 can receive friction pegs 46, the third article retention pouch 26 can receive and retain a mouth call 48, and the first article retention pouch 14 can receive and retain a call member 44.

In a first mode of using the third embodiment 30 of the invention, as shown in FIGS. 6 and 7, the combination of the first, second, and third pouches are positioned on the glove back side, and it is locked on the glove back side by the lock tab 50, such that the hook-or-loop connector 52 on the lock tab 50 engages a complementary loop-or-hook connector on the glove portion 12.

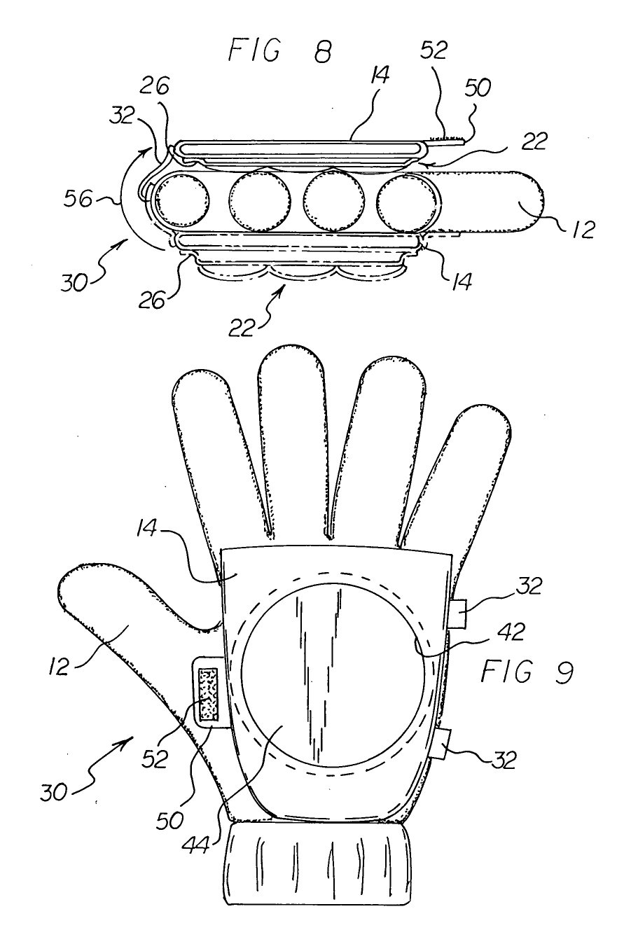

In a second mode of using the third embodiment 30 of the invention, as shown in FIGS. 8 and 9, the lock tab 50 is disengaged from the glove portion 12, and the combination of the first, second, and third pouches is rotated around the hinge rotation regions 54 on the flexible hinge members 32, as shown by rotational arrow 56, so that the combination of the first, second, and third pouches is moved from the glove back side to the glove palm side. The position of the combination of the first, second, and third pouches on the glove back side is shown in broken lines in FIG. 8. The position of the combination of the first, second, and third pouches on the glove palm side is shown in solid lines in FIG. 8.

Once the combination of the first, second, and third pouches is resting on the glove palm side, the opening in the first article retention pouch 14 provided by the elastic frame 42 permits the call member 44 to be contacted directly by one or more of the friction pegs 46 to make a calling sound for game, without removing the call member 44 from the first article retention pouch 14.

When it is no longer needed to have the combination of the first, second, and third pouches on the glove palm side, the combination of the first, second, and third pouches is rotated on the flexible hinge members 32 around the hinge rotation regions 54 in the opposite direction so that the combination of the first, second, and third pouches is positioned again on the glove back side. Then, the lock tab 50 is used to lock the combination of the first, second, and third pouches to the glove back side.

The shifting of the combination of the first, second, and third pouches around the flexible hinge members 32 can be thought of as flipping the combination of the first, second, and third pouches around from the glove back side to the glove palm side and vice versa. With this embodiment of the invention, the combination of the first, second, and third pouches is a flip pouch.

The glove portion 12 can be in a variety of forms, including mesh or full cloth material. The respective pouches in the combination of the first, second, and third pouches can be made of a solid covering material in order to keep moisture out.

The call member 44 can be in a variety of forms, for example slate. The call member 44 can take the form of a variety of turkey calls or other game calls.

Alternatively, the glove-to-retainer connection means can includes a zipper. Also, alternatively, the lock means between the combination of the first, second, and third pouches and the glove portion 12 can includes snap buttons.

The components of the game call retention apparatus of the invention can be made from inexpensive and durable cloth and plastic materials.

As to the manner of usage and operation of the instant invention, the same is apparent from the above disclosure, and accordingly, no further discussion relative to the manner of usage and operation need be provided.

It is apparent from the above that the present invention accomplishes all of the objects set forth by providing a new and improved game call retention apparatus that is low in cost, relatively simple in design and operation, and which may advantageously be used to enable a hunter to access a game call apparatus without removing one's gloves. With the invention, a game call retention apparatus is provided which can retain a plurality of different types of game call apparatuses. With the invention, a game call retention apparatus is provided which can be readily separated from the hunter. With the invention, a game call retention apparatus is provided which precludes the need for a hunter to store a game call apparatus in a conventional garment pocket. With the invention, a game call retention apparatus is provided which is located in the close vicinity of the hunter's hands. With the invention, a game call retention apparatus is provided which enables a hunter to use the game call retention apparatus without removing the game call apparatus from the retention apparatus.

Thus, while the present invention has been shown in the drawings and fully described above with particularity and detail in connection with what is presently deemed to be the most practical and preferred embodiment(s) of the invention, it will be apparent to those of ordinary skill in the art that many modifications thereof may be made without departing from the principles and concepts set forth herein, including, but not limited to, variations in size, materials, shape, form, function and manner of operation, assembly and use.

Hence, the proper scope of the present invention should be determined only by the broadest interpretation of the appended claims so as to encompass all such modifications as well as all relationships equivalent to those illustrated in the drawings and described in the specification.

Finally, it will be appreciated that the purpose of the annexed Abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. Accordingly, the Abstract is neither intended to define the invention or the application, which only is measured by the claims, nor is it intended to be limiting as to the scope of the invention in any way.

1. An article retention apparatus, comprising:
a glove portion,
first article retention means which include first retainer closure means, and
glove-to-retainer connection means for connecting said glove portion to said first article retention means,
wherein said first article retention means include a first article retention pouch so constructed and arranged to retain therein a game call member capable of being sounded when hit or scratched by a friction member.

2. The apparatus of claim 1 wherein said glove-to-retainer connection means are located so as to enable positioning of said first article retention means on a back side of said glove portion.

3. The apparatus of claim 1 wherein said glove-to-retainer connection means include stitches between said first article retention means and said back side of said glove portion.

4. The apparatus of claim 1 wherein:
said first retainer closure means include a first retainer closure elastic band located at an opening of said first article retention pouch.

5. The apparatus of claim 1 wherein said first article retention pouch includes an elastic frame for peripherally retaining said call member to permit said call member to be exposed for striking by said friction member when said call member is retained thereby.

6. The apparatus of claim 4 wherein said glove-to-retainer connection means include:
a pouch-mounted hook-or-loop connector attached to a back side of said first article retention pouch, and
a complementary glove-mounted loop-or-hook connector attached to said glove back side.

7. The apparatus of claim 1 wherein said first retainer closure means is made from closure hook-or-loop material.

8. The apparatus of claim 1, further including:
second article retention means connected to said first article retention means.

9. The apparatus of claim 8 wherein said second article retention means include a second article retention pouch so constructed and arranged as to retain therein said friction member.

10. The apparatus of claim 9 wherein said second article retention pouch includes a plurality of pouch compartments.

11. The apparatus of claim 10 wherein said pouch compartments include an open top end and an open bottom end.

12. The apparatus of claim 9 further including:
a third article retention pouch connected between said first article retention pouch and said second article retention pouch,
wherein said third article retention pouch is so constructed and arranged to retain therein a game call member in the form of a mouth call.

13. The apparatus of claim 8 wherein said glove-to-retainer connection means include flexible hinge members connected between said glove portion and said first article retention pouch.

14. The apparatus of claim 13 wherein said flexible hinge members are connected to said glove portion at hinge rotation regions.

15. The apparatus of claim 14 wherein said flexible hinge members are connected to a side edge of said glove portion.

16. The apparatus of claim 8, further including:
lock means for locking said first article retention pouch to a back side of said glove portion.

17. The apparatus of claim 16 wherein said lock means include a lock tab.

18. The apparatus of claim 17 wherein said lock tab includes a quantity of a hook-or-loop connector which engages a quantity of complementary loop-or-hook connector on said glove portion for locking said lock tab to said glove portion.
